(19) United States
(12) Patent Application Publication
(10) Pub. No.: US 2024/0065981 A1
(43) Pub. Date: Feb. 29, 2024

(54) BORONATED NANOSCALE SUBSTRATE AND USES THEREOF

(71) Applicant: San Jose State University, San Jose, CA (US)

(72) Inventors: Abraham Wolcott, Scotts Valley, CA (US); Krishna Govindaraju, Santa Clara, CA (US); Sandra Ladjadj, San Jose, CA (US); Daniel Labunsky, San Jose, CA (US); Ezhioghode Uwadiale, San Jose, CA (US)

(21) Appl. No.: 18/454,139

(22) Filed: Aug. 23, 2023

Related U.S. Application Data
(60) Provisional application No. 63/400,256, filed on Aug. 23, 2022.

(51) Int. Cl. A61K 9/127

(52) U.S. Cl. CPC A61K 9/1277; A61K 9/1271; B82Y 5/00

(57) ABSTRACT

Various aspects described herein relate to a functionalized nanoscale substrate. The functionalized nanoscale substrate includes a functionalized surface of the substrate that includes a boronated portion.

### CLAIM OF PRIORITY

This patent application claims the benefit of priority to U.S. Application Ser. No. 63/400,256, filed Aug. 23, 2022, which is incorporated by reference herein in its entirety.

### STATEMENT OF GOVERNMENT SUPPORT

This invention was made with government support under W911NF-18-1-0453 awarded by the Army Research Laboratory—Army Research Office, and 1SC3GM125574-01 awarded by the National Institutes of Health. The U.S. Government has certain rights in this invention.

### BACKGROUND

Nanoscale particles are suited to many different uses. The use and efficacy of these nanoscale particles can be controlled by functionalizing the nanoparticles.

### SUMMARY OF THE INVENTION

Various aspects described herein relate to a functionalized nanoscale substrate. The functionalized nanoscale substrate includes a functionalized surface of the substrate that includes a boronated portion.

### BRIEF DESCRIPTION OF THE DRAWINGS

The drawings illustrate generally, by way of example, but not by way of limitation, various aspects of the present invention.

FIG. 1A is a Transmission Electron Microscopy (TEM) image of DND nanocrystals. Average crystal size of diamonds created by this process is 4-6 nm.

FIG. 1B is a TEM image of synthetic HPHT diamond nanocrystals. The size of these of these diamonds can vary from less than 5 nm to as large as 500 nm.

FIG. 1C is a macroscale image of diamonds fabricated via chemical vapor deposition.

FIG. 2 is a schematic image showing Diamond cubic crystalline structure pictured with all possible Nitrogen vacancy sites. Sites are classified based on the orientation of their symmetry axes. Carbon is denoted by the solid atoms while Nitrogen is indicated by hatched atoms.

FIG. 3 is a schematic image showing illustrating radiochemotherapy using Boron-containing agents as targets for irradiation. One neutron reacts with a Boron-10 nucleus to produce Lithium-7 and Helium-4.

FIG. 4 is a schematic diagram of Boron nanoparticles encapsulated in a liposomal structure and functionalized at the surface with different ligands.

FIG. 5 is a graph showing fourier transform infrared spectroscopy of organoborane functionalized NDs. Samples were reacted for 180 min with various trigonal boron precursors including BBr₃, BCl₃ and BH3. All samples exhibit a characteristic shift from 1105 wavenumbers to 1025 wavenumbers indicative of C—B bonds.

FIG. 6 is a graph showing fourier transform infrared spectroscopy of organoborane functionalized NDs. Samples were reacted for 24 hours with various trigonal boron precursors including BBr₃, BCl₃ and BH₃. All samples exhibit a characteristic shift from 1105 wavenumbers to 1025 wavenumbers indicative of C—B bonds.

FIG. 7 is an SEM image of unreacted HPHT nanodiamond control.

FIG. 8 is an image of nanodiamond cores after boron templating indicates morphology changes following synthetic protocol with trigonal boron precursors.

FIG. 9 is an image showing SEM and EDS data were collected on boron carbide.

FIG. 10 is an EDS scan is for a ND—OH sample reacted with BBr₃ in DCM.

FIG. 11 is a graph showing X-ray absorption spectroscopy shows core hole excitation at 289 eV and 2nd absolute bandgap at 305 eV.

FIG. 12 is a graph showing X-ray photoelectric spectroscopy of organoborane functionalized NDs.

FIG. 13 is a graph showing liposome fabrication resulted in polydisperse multilamellar structures.

FIG. 14 is a graph showing liposome fabrication with BNDs resulted in polydisperse multilamellar structures before purification that were analyzed using DLS.

FIG. 15 is a scanning confocal microscopy image of Lucifer yellow control dissolved in chloroform at 40× objective

FIG. 16 is a scanning confocal microscopy images of Liposomes with boronated NDOH at 40×.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

Various aspects of the disclosure described herein relate to a functionalized nanoscale substrate. The substrate includes a boronated portion that defines a functionalized surface. Generally, the boronated portion is a ¹⁰B boron. The substrate is a nanoscale particle or nanoparticle that can include diamond, gold, silver, silica or a mixture thereof. In certain aspects the substrate includes diamond. A major dimension of the nanoscale substrate can be in a range of from about 5 to about 100 nm or about 15 to about 80 nm.

The boronated portion can be disposed over about 40 to about 100 percent of a total surface area of the surface of the substrate or about 60 to about 100 percent of a total surface area of the surface of the substrate.

The functionalized nanoscale substrate can be incorporated into a delivery vehicle. The delivery vehicle can include a liposome encapsulating the functionalized nanoscale substrate described herein.

As described further herein the functionalized nanoscale substrate can be formed in general by boronating a hydroxylated nanoscale substrate. Boranating the substrate can include reacting the hydroxylated nanoscale substrate with boron tribromide, boron trichloride, or a mixture thereof. The delivery vehicle can be formed by contacting the functionalized nanoscale substrate with one or more lipids such as a phospholipid. Then the functionalized nanoscale substrate can be mixed with the one or more lipids to form one or more liposomes encapsulating the functionalized nanoscale substrate.

The functionalized nanoscale substrates can be used to treat a tumor. A method of treatment includes administering a therapeutically effective amount of the functionalized nanoscale substrate to a patient in need thereof. The method further includes contacting the administered functionalized nanoscale substrate with electromagnetic radiation. Examples of tumors that can be treated include a brain tumor. More specifically, tumors that can be treated include a cutaneous melanoma or a glioblastoma.

Carboranes and organo-metalloids have shown great promise in the field of medicinal chemistry. Recently, Carboranes have been utilized in drug design for their ability to instantly convert from trigonal planar configuration (sp2) to a more neutral or tetrahedral configuration (sp3) due to the electron deficient nature of carborane complexes. More recently, advancements in the field of nuclear medicine have shown that carboranes could be useful for Boron neutron capture therapy (BNCT). Thermal neutron capture of Boron-10 induces a fission reaction releasing Lithium-7 (⁷Li) and alpha-particles (Helium-4 or ⁴He) along with gamma radiation that can be used to treat cancerous tissue. Currently, there are a number of factors that hinder the ability for BNCT to be used safely and effectively. For the therapy to be effective, an appropriate number of ¹⁰B atoms must be introduced to the neoplastic cells at an appropriate concentration, then irradiated only once integration of the carborane complex into the cell has been achieved.

A surface modification of hydroxyl-terminated fluorescent nanodiamonds with Boron-10 (¹⁰B) is proposed as a treatment for cutaneous melanomas, glioblastomas and other head and neck cancers using neutron capture therapy. There is a need for clinicians to be able to properly localize ¹⁰B to the tumor site in appropriate concentrations to improve the specificity of radiation therapy and targeted destruction of cancer cells. Modified oxidized nanodiamonds could be reacted with trigonal boron precursors to produce a boron rich surface coating. These modified diamonds could act as novel delivery vectors for ¹⁰B and therefore be targets for thermal neutron capture. It has been reported that a cell requires an average of 3-7 alpha particles to destroy cancerous tissue about 15 μg in size. Functionalization of the surface of the nanodiamonds with ¹⁰B could allow for adequate concentrations to enter the neoplastic cells to be targeted for irradiation and ultimately improve the viability of this therapy. Nanodiamonds have yet to be explored as a potential substrate for a ¹⁰B and could replace the polyhedral boron clusters that have been used in the past as vectors for radioactive boron in cancer treatment. Ultimately, these nanomaterials could potentially address clinicians’ need for a robust and non-toxic carrier for Boron in a variety of medical applications.

Functionalization of the NV nanodiamonds begins with oxidization of the nanodiamond surface to improve reactivity by introducing hydroxyl terminations at the surface. From here, trigonal planar Boron compounds (BH₃, BCl₃, or BBr₃) act as electrophiles and react with the tertiary alcohols at the oxidized ND surface. The electron-rich hydroxyl groups at the diamond surface interact directly with the electron-deficient boron compounds to produce a shell of boron around the diamond core. This shell is highly reactive, and its size is hypothesized to be able to be controlled by modulating the amount of time electrophiles are allowed to react with the oxidized diamonds or by increasing the concentration of electrophile used; after purification and removal of by-products, the functionalized samples are analyzed to characterize the extent of boron templating.

In order to improve localization of the ¹⁰B coated nanodiamonds to the site of interest, liposomes have been proposed as a way to encapsulate and safely deliver functionalized nanodiamonds directly to the tumor site. Liposomes are essentially vesicles of amphiprotic phospholipids that form a circular membrane or micelle which can mimic a cancerous cell's membrane. Another advantage of using liposomes to deliver the boronated diamonds to the tumor site is that liposomes can be further functionalized at the surface to improve their specificity to neoplastic cancer cells.

Diamond samples are analyzed using scanning electron microscopy (SEM), Fourier transformed Infrared spectroscopy (FTIR), Energy deficient X-ray spectroscopy (EDS) and X-ray photoelectric spectroscopy (XPS) to confirm that boronation has indeed occurred at the diamond surface. Liposomes are then fabricated around functionalized particles and visualized using confocal microscopy, and other fluorescent microscopy techniques. In an effort to improve the viability of BNCT, the functionalized nanodiamonds and liposomes are characterized and visualized of help verify that these protocols are successful in fabricating effective vectors for ¹⁰B delivery.

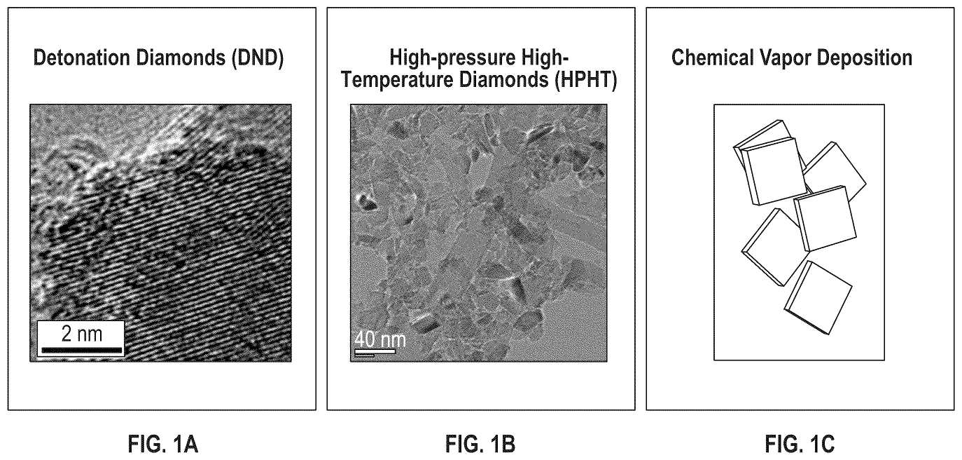

Carbon based nanomaterials are versatile materials favored for their biocompatibility, chemical inertness and unique thermochemical properties. They have been used for drug delivery, tissue engineering, tissue scaffolding, biosensing and various other fields of biomedical research. Nanodiamonds (NDs) exist as a tetrahedral carbon lattice imparting them with highly desirable physical and chemical properties. Carbons within the lattice are in the sp³ configuration. The surface of the tetrahedral structures can be oxidized to create alcohol-rich surface terminations that can improve reactivity and allow for functionalization of the nanoparticle. There are many ways to produce NDs including chemical vapor deposition, applying high temperature high pressure (HTHP), or detonating explosives. The average size of diamond nanoparticles can range from 5-100 nm depending on how the materials are synthesized or isolated (FIG. 1). Mechanically, NDs have a higher toughness and modulus of elasticity when compared to macro-scale diamonds, or other nanoparticles used for biomedical applications such as gold and silver. At the nanoscale, the ND sp³ bonding configuration makes them similar to macroscale diamonds in terms of chemical stability and thermal conductivity. These material properties ensure the nanodiamond particles are resistant to fractures; they are also electrically resistive with a high refractive index further improving their utility as optical sensors or drug delivery vectors. NDs are particularly interesting materials for two reasons: they are small enough to be useful in a variety of biological and nanoscale environments, and they can be selectively modified at the surface to enhance the NDs electrochemical and thermal conductive properties.

Atomic defects, more specifically the Nitrogen Vacancy (NV), allow scientists to record energy transfers and detect small transfers of energy between molecules in vitro. NV diamonds are naturally fluorescent, absorbing photons in the green range of visible light and emitting a detectable signal in the red range. Light is used to selectively excite one molecule and used to detect the electromagnetic environment (within 1-10 nanometers) by donating a photon to an accepting fluorophore. Energy transfers can then be measured by an instrument such as an electron multiplying charge coupled device (EMCCD) which detects a changing fluorescent signal. Advances in the field of microscopy have allowed for major breakthroughs in the utility of carbon nanodiamonds as functional biosensors and targets for optically detectable magnetic resonance (ODMR). Although many different biophysical techniques can be used to assess magnetic resonance, robust ODMR provides data that is useful for the study of single molecule fluorescence resonance energy transfer (smFRET) events such as ligand binding or conformational changes in biopolymers with high specificity. ODMR measurements can be time dependent and spin readout can be induced and detected using a fluorescent microscope. When the NV defect is negatively charged and stabilized by a hydroxyl termination at the surface, an electron spin readout can be obtained after stimulation with photons. When compared to traditional continuous wave ¹H nuclear magnetic resonance (¹HNMR). There has been evidence to suggest that smFRET can measure nanoscale distances between complexes that exchange energy radiatively. Through characterization of modified fluorescent nanodiamonds, we hope to enhance our understanding and optimize the performance of these nanomaterials for biomedical applications and single molecule experiments.

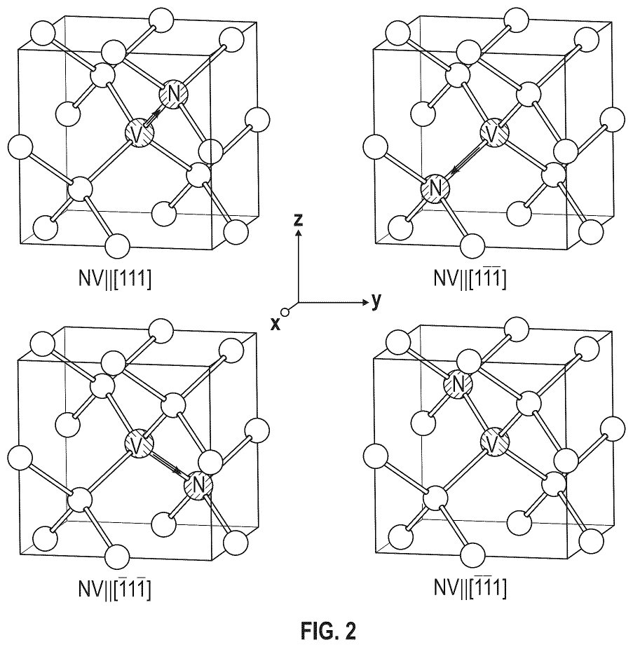

Nitrogen vacancy (NV) defects consist of a missing carbon within the diamond lattice structure, which is immediately adjacent to a substituted nitrogen (FIG. 2). These NV centers are sensitive to externally applied magnetic fields which make them useful for non-invasively measuring electromagnetic fields in delicate tissues such as the brain. The fluorescent signal from NV is extremely stable and remarkably resistant to photobleaching or signal intermittency. In recent years, as more data reveals the importance of the NV in quantum information processing, there has been a substantial effort to control the charge of the NV centers. The negatively charge state of the NV center hosts a long-lived electron spin state that coheres to its electrostatic environment making it useful as an electromagnetic probe. To this end studies have been conducted to try and improve the stability of the negatively NV charge centers. In the past, researchers have observed that NV centers will spontaneously transition between the ground state (NV⁰) and charged state (NV⁻) when exposed to a continuous photon source such as a laser. In order to improve the stability of the NV, a potential difference is applied to the surface of the diamond to force NV⁰ centers to an excited state. Finally, the NDs can be chemically modified at the surface to improve the NV⁻ stability.

NDs that have naturally occurring NV impurities can form complexes at the surface with a variety of ligands including peptides, amines, silanes and metalloids; such ND chemistry will be the focus of our research. NDs holding NV centers provide fluorescent properties, impart special optical properties and can be enhanced or modulated with selective functionalization of the ND surface. Certain modifications can act to enhance the fluorescent signal emitted by the defect, modulate colloidal stability or directly affect the ground state stability of the NV. Modification at the surface of the NDs with metalloids including Boron was explored in an effort to improve reactivity of the diamond surface due to high steric hinderance and chemical inertness. Boron often aggregates to form 3-center 2-electron pair structures because organoborane chemistry is characterized by delocalized electron deficient bonding. NDs are oxidized at high temperatures to provide an alcohol-rich surface. The oxidized nanodiamond can then be reacted with Boron compounds to form metalloid complexes at the diamond surface.

Boron Neutron Capture Therapy

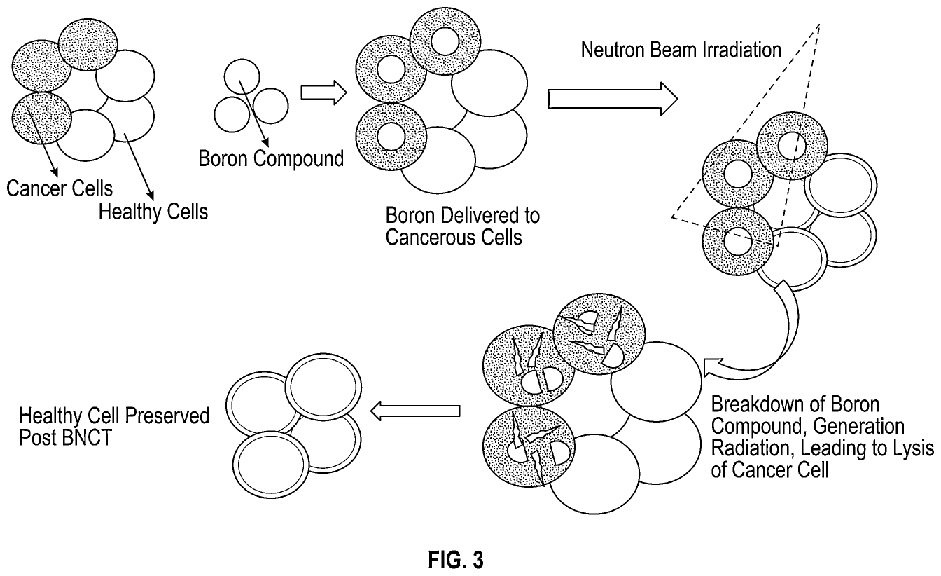

Although coating the surface of the diamond with Boron is useful for performing further chemistry, it may also hold therapeutic benefits. Boron neutron capture as a radiotherapy has been explored as a possible treatment for aggressive brain tumors since neutrons were discovered in the 1930's. Boron-10 was of specific interest to clinicians due to the highly localized nature of the fission reaction that occurs when ¹⁰B is irradiated with a low-energy neutron beam (FIG. 3). As medical science and pharmaceutical chemistry continued to advance, new Boron compounds were synthesized and BNCT became more feasible. By 1968, the therapy had successfully been used in Japan with 54% of some glioblastoma patients in a study surviving past 5 years once beginning treatment. In the past, there were very few first-generation Boron agents all of which were chemically unstable, prone to degradation or exhibited poor tumor retention. Boron carries were injected directly into the tumor site following surgical ablation and irradiated with moderate success. With the advent of third generation Boron compounds, the procedure instead involves intravenously or intrathecally administering various Boron carriers, then administering radiation without the need for immediate surgery. To minimize the amount of vascular tissue that is damaged by the irradiation, the amount of therapeutic agent in the tumor mass is carefully considered in relation to the amount of agent in the surrounding tissue and blood. Generally, the original Boron target is radioactively labeled and tracked using positron emission tomography to ensure that it has localized to the tumor. It is estimated that approximately ¹⁰B atoms are needed to irradiate each cell or approximately 30 mg ¹⁰B for every gram of tumor mass. The overall equation that governs the principles of BNCT is:

¹⁰B+¹n→[¹¹B]*→⁴He+⁷Li(2.31MeV)+γ(0.48MeV)

Once excited by a thermal neutron, the excited ¹¹B nucleus decays by fission to yield an alpha particle and a recoiling Lithium nucleus with a small amount of gamma radiation; this radiation has a range between 5-10 μm making it useful for specifically targeting tumors and leaving the surrounding vasculature intact.

Boron chemoradiotherapies such as BNCT use polyhedral carborane structures for delivering ¹⁰B to the tumor site in sufficient quantities. In the past, scientists used first-generation inorganic borates such as borax and boric acid. These agents were considered unsuitable for radiochemotherapy due to the fact that these compounds were not very specific for the tumors, and often did not persist in once introduced. Polyhedral boranes, more specifically icosahedral dicarba-closo-dodecaboranes, are relatively non-toxic and pack a large amount of Boron into a small volume making them agreeable candidates for irradiation. An ideal target for BNCT is easily capable of crossing cell membranes, crossing the blood brain barrier (BBB), and persisting in the cell by evading catabolic enzymes. More recently, there has been great interest in using Boron-containing compounds such as para-borophenylalanine (BPA) and sodium mercaptoundecahydrododecaborate (BSH). BPA strongly resembles the amino acid phenylalanine as well as the pigment melanin, and is easily allowed to infiltrate the tumor mass. BSH is another polyhedral borate observed to accumulate very well in tumors but not in the surrounding tissues. These so-called 2^{nd} generation Boron compounds are allowed to bioaccumulate and incorporate themselves into cellular substructures eventually becoming robust targets for irradiation and subsequent destruction. Aside from BPA and BSH, very few Boron agents have made it past animal studies and clinical trials. The lack of knowledge surrounding Boron nanoparticles makes metalloid surface modifications of diamond especially interesting to study.

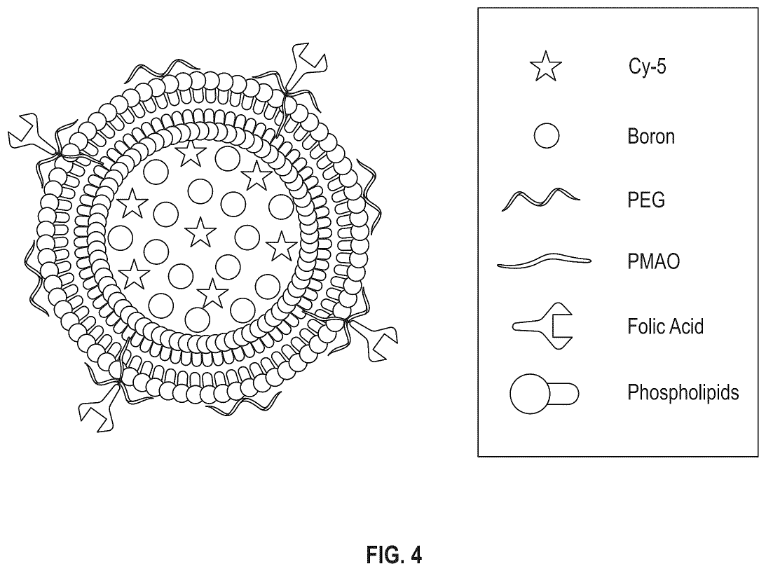

One of the drawbacks to using Boron clusters such as decaborane and dodecaborane, is that the synthesis of these compounds is often dangerous and expensive. Conventional synthesis of Boron clusters or other Boron hydrides either involves combining Boron trifluoride with Sodium borohydride or the pyrolysis of diborane gas. For this reason, specialized equipment is needed to handle the materials or perform syntheses on an industrial scale. By and large, the biggest issue is the lack of an efficient transport mechanism to bring the activated ¹⁰B compound from the site of administration to the site of action. This is further complicated by diverse cancer cell subpopulations that may uptake the Boron agent variably resulting in persistence of the cancerous tissue. To improve the viability of BNCT, alternatives to polyhedral borane clusters must be considered as drug delivery substrates. Nanodiamond is a durable substrate with a high surface-to-volume ratio that could potentially deliver a large number of ¹⁰B atoms to an intended target. To further address the biggest issue faced with currently accepted Boron drugs or carriers, the functionalized nanocrystals can be encapsulated in a vesicle or liposome (FIG. 4). Liposomes could guide the NDs safely across the BBB and specifically target surface receptors on the cancer cells while avoiding healthy tissues and evading the immune system. In this way, Boron can be introduced via ND into a patient's body intravenously and travel directly to the brain to directly target neoplastic cells.

Liposome Chemistry

Liposomes are some of the most successful vectors for drug delivery ever discovered. Fundamentally, their structure is fairly simple consisting of a simple phospholipid bilayer. This thin membrane consists of hydrophilic outer and inner walls, with a hydrophobic core (FIG. 4). One of their best utilities as biocarriers is their ability to be optimized at the surface for specific functions. For example, in most drug delivery applications a small amount of polyethylene glycol (PEG) can be attached to the polar outer surface of the liposome to help it evade opsonization or phagocytosis. Cholesterol or other steroid molecules can be added to control the fluidity of the membrane and stabilize the hydrophobic core of the liposomal core. Simple modifications can be made to the surface to improve tumor specificity; in one study, folic acid was added to the liposome surface because it was found that many tumor cells tend to overexpress folic acid receptors at the cell membrane. Localization studies in vitro showed that folic acid functionalized liposomes infiltrated the BBB model more effectively than liposomes that were not coated with folic acid.

Fabricating liposomes can be approached in many ways and largely depends on the drug contents to be loaded, solvent in which the liposomes are dispersed, and the scale of production. Scientists also consider the type of vesicle required for the given application. Unilamellar structures consist of a typical phospholipid shell with an aqueous core, while multilamellar structures consist of one or more concentric spheres. For the purpose of encapsulating Boron nanoparticles, unilamellar structures are favorable due to the fact that they more effectively hold hydrophilic contents than multilamellar species. A common way to prepare small batch liposome micelles is via thin-film hydration. This technique involves dispersion of the phospholipid in an organic solvent, and rotary evaporation of the organic solvent to obtain a thin lipid film. Finally, the thin film is rehydrated in an aqueous buffer solution. Thin-film hydration often produces many multilamellar liposomes that require tedious extrusion and sonication to become homogenous and unilamellar. Reverse-phase evaporation and solvent injection methods are alternative ways to fabricate liposomes. These processes work by hydrating phospholipids directly from an organic solvent to yield an aqueous suspension containing multilamellar or unilamellar vesicles depending on the process used (Reverse-phase evaporation yields multilamellar species, while solvent injection yields unilamellar species). Once an ideal liposome fabrication methodology is selected, it should be determined in what solvent the drug contents, or Boron-coated NDs, will be in before it is loaded into the liposome micelles. Lipophilic drugs or strongly non-polar solvents can be combined with the lipid film prior to hydration. Alternatively, hydrophilic drugs can be added to the aqueous solution used to hydrate the lipid film. Finally, once liposome species have been appropriately labeled and drug loaded, they can be visualized using different microscopy techniques and further characterized through spectroscopic analysis.

Fabricating boron nanostructures to better control their delivery for therapeutic applications has been instrumental in improving the utility and efficacy of boron neutron capture therapy. Traditionally, pyrolysis of diborane gas using a photon emission source has been used to synthesize boron nanoparticles. A significant drawback of this method of synthesis is that laser heating of diborane gas to temperatures greater than 1000° C. produces amorphous boron nanostructures with a wide size distribution. To reduce the size distribution of boron particles, arc decomposition of diborane gas is favored because it greatly reduces the number of amorphous structures by producing particles between 55 and 95 nm. Polyhedral Boron and carborane clusters have also been fabricated for the purpose of facilitating neutron capture therapy. Transition-metal catalysts are used in template-assisted self-assembly for creating block copolymers via pyrolysis of diborane gas. An alternative method focuses on multiphase solution-phase synthesis. Solution-phase synthesis requires commercial boron powder to be wet milled and subsequently functionalized with sodium dodecyl sulfate (SDS) or oleic acid to reduce air oxidation and prevent autoignition of nanoscale boron particles. An air-free methodology for Boron nanoparticle synthesis has been proposed to reduce the need for gas-phase decomposition of diborane gas; solution synthesis of organo-capped boron nanoparticles is reduced via Boron Tribromide with sodium naphthalenide in dry dimethoxyethane. This method eliminates the need for the use of the flammable and toxic gas phase diborane. Here, we propose a novel method for surface functionalization of carbon nanodiamonds through the formation of carborane structures via reduction of oxidized nanodiamonds with Boron Tribromide and other reactive Boron Trihalides.

If oxidized nanodiamonds are reacted with trigonal boron compounds under inert conditions, the hydroxyl terminations will act as nucleophiles and form stable organoborate structures at the diamond surface.

If boron can be safely templated onto the surface of nanodiamonds, they can be loaded into liposomal structures without affecting the surface chemistry of the boron-nanodiamond structures.

If it is possible template trigonal boron structures onto carbon nanocrystals, it should be theoretically possible to use the same protocol to template boron onto other nanoparticles such as gold or silica.

According to various aspects of the instant disclosure a functionalized nanoscale substrate includes a functionalized surface of the substrate. At least a portion of the functionalized surface is a boronated portion. The substrate can include any suitable material. Examples of suitable materials include diamond, gold, silver, silica or a mixture thereof. According to various aspects, diamond is a particularly well suited substrate material. The boronated portion includes a ¹⁰B boron.

The boronated portion is disposed over about 40 to about 100 percent of a total surface area of the surface of the substrate, about 60 to about 100 percent of a total surface area of the surface of the substrate, less than, equal to, or greater than about 40 percent of a total surface area of the surface of the substrate, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 percent of a total surface area of the surface of the substrate.

According to some examples, the functionalized nanoscale substrate is encapsulated by a liposome. A liposome has an aqueous solution core surrounded by a hydrophobic membrane, in the form of a lipid bilayer; hydrophilic solutes dissolved in the core cannot readily pass through the bilayer. Hydrophobic chemicals associate with the bilayer. This property can be utilized to load liposomes with hydrophobic and/or hydrophilic molecules, a process known as encapsulation. Typically, liposomes are prepared in a solution containing the compound to be trapped, which can either be an aqueous solution for encapsulating hydrophilic compounds like proteins, or solutions in organic solvents mixed with lipids for encapsulating hydrophobic molecules. Encapsulation techniques can be categorized into two types: passive, which relies on the stochastic trapping of molecules during liposome formation, and active, which relies on the presence of charged lipids or transmembrane ion gradients. A crucial parameter to consider is the “encapsulation efficiency,” which is defined as the amount of compound present in the liposome solution divided by the total initial amount of compound used during the preparation. In more recent developments, the application of liposomes in single-molecule experiments has introduced the concept of “single entity encapsulation efficiency.” This term refers to the probability of a specific liposome containing the required number of copies of the compound.

To deliver the molecules to a site of action, the lipid bilayer can fuse with other bilayers such as the cell membrane, thus delivering the liposome contents; this is a complex and non-spontaneous event, however, that does not apply to nutrients and drug delivery. By preparing liposomes in a solution of DNA or drugs (which would normally be unable to diffuse through the membrane) they can be (indiscriminately) delivered past the lipid bilayer. Liposomes can also be designed to deliver drugs in other ways. Liposomes that contain low (or high) pH can be constructed such that dissolved aqueous drugs will be charged in solution (i.e., the pH is outside the drug's pH range). As the pH naturally neutralizes within the liposome (protons can pass through some membranes), the drug will also be neutralized, allowing it to freely pass through a membrane. These liposomes work to deliver drug by diffusion rather than by direct cell fusion. However, the efficacy of this pH regulated passage depends on the physiochemical nature of the drug in question (e.g. pKa and having a basic or acid nature), which is very low for many drugs.

According to various aspects of the instant disclosure a therapeutically effective amount of the functionalized nanoscale substrate can be administered to a patient in need thereof. The functionalized nanoscale substrate can be encapsulated in the liposome as a pharmaceutical composition or kit. A “therapeutically effective amount” (or “effective amount”) of a compound with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the patient of one or more compound of the disclosure. If it is administered prior to clinical manifestation of the unwanted condition (e.g., disease or other unwanted state of the host animal) then the treatment is prophylactic, (e.g., it protects the host against developing the unwanted condition), whereas if it is administered after manifestation of the unwanted condition, the treatment is therapeutic, (e.g., it is intended to diminish, ameliorate, or stabilize the existing unwanted condition or side effects thereof).

As used herein, the term “kit” refers to a product (e.g. medicament, kit-of-parts) comprising one package or one or more separate packages of:
(i). A pharmaceutical composition containing an active pharmaceutical ingredient and at least one further active pharmaceutical ingredient and optionally a medical device. The at least one further active pharmaceutical ingredient may be present in said pharmaceutical composition, i.e. the kit may comprise one or more packages, wherein each package comprises one pharmaceutical composition which comprises two or more active pharmaceutical ingredients. The further active pharmaceutical ingredient may also be present in a further pharmaceutical composition, i.e. the kit may comprise separate packages of two or more pharmaceutical compositions, wherein each pharmaceutical composition contain one active pharmaceutical ingredient.

### Or

(ii). A pharmaceutical composition containing an active pharmaceutical ingredient and medical device.

A kit may comprise one package only or may comprise one or more separate packages. For example, the kit may be a product (e.g. medicament) containing two or more vials each containing a defined pharmaceutical composition, wherein each pharmaceutical composition contains at least one active pharmaceutical ingredient. For example, the kit may comprise (i.) a vial containing a defined pharmaceutical composition and (ii). further a tablet, capsule, powder or any other oral dosage form which contains at least one further active pharmaceutical ingredient. The kit may further comprise a package leaflet with instructions for how to administer the pharmaceutical composition and the at least one further active pharmaceutical ingredient.

As used herein, the term “medical device” means any instrument, apparatus, implant, in vitro reagent or similar or related article that is used to diagnose, prevent, or treat a disease of other condition, and does not achieve its purpose through pharmacological action within or on the body.

As used herein, a medical device may be a syringe, an insulin injection system, an insulin infusion system, an insulin pump or an insulin pen injection device. As used herein, a medical device may be mechanically or electromechanically driven.

The ingredients in the pharmaceutical composition can be defined as being Generally Recognized as Safe (“GRAS”). A full list of GRAS ingredients can be found in the GRAS Substances (SCOGS) Database maintained by the United States Food and Drug Administration. About 50% to about 100% of the ingredients in the pharmaceutical composition can be classified as being GRAS ingredients, about 75% to about 100%, about 90% to about 100%, less than, equal to, or greater than about 50%, 55, 60, 65, 70, 75, 80, 85, 90, 95, or about 100% of the ingredients in the pharmaceutical composition can be classified as being GRAS ingredients.

Following administration, the functionalized nanoscale substrate is irradiated with electromagnetic radiation. The functionalized nanoscale substrate is not irradiated with electromagnetic radiation until the substrate is proximate to a tumor. The tumor can be a brain tumor. Examples of brain tumors include a cutaneous melanoma or a glioblastoma.

### Examples

Materials & Methods

High-pressure high-temperature nanodiamond powders (monocrystalline diamond powder, MSY 0-0.03 micron and MSY 0-0.05 micron) will be purchased from Microdiamant, USA. Anhydrous dichloromethane (≥99.8% Product #270997), boron tribromide (≥99% Product #230367), phenylboronic acid pinacol ester (97% #647098), trans-1-propenylboronic acid pinacol ester (97% #735558), triethyl borane (1.0 M in tetrahydrofuran Product #179701), triphenyl borane (0.25 M in tetrahydrofuran #442445), boron trichloride (1.0 M in methylene chloride #178934), trimethyl borate (≥99% #92330), triethyl borate (99% #T59307), tributyl borate (≥99% #90795), tri-tert-butyl borate (98% #236608), Ammonia (0.4 M in tetrahydrofuran #718939), 1,1,1,3,3,3-Hexafluoro-2-propanol (≥99% #105228), Propargylamine (98% #P50900) will be purchased from Sigma Aldrich (St. Louis, MO). Boron carbide powder (95% 42 μm #815-96) and 400 mesh, Copper, TEM Grids with Ultrathin Lacey Carbon Film (#01824) are purchased from Ted Pella, Inc. (Redding, CA). Boron powder (95% #47303) is purchased from Alfa Inorganics (Ward Hill, MA). 4-inch silicon wafers coated with a 10 nm titanium adhesion layer and 100 nm of gold will be purchased from LGA Thin Films, Inc. (Santa Clara, CA). Spectra Tech, Potassium Bromide powder packets (#0016031) will be purchased from Thermo Fisher Scientific (Waltham, MA).

A Thermo Scientific (STF55346COMC-1) three-zone tube furnace is used to aerobically oxidize −30 and 50 nm HPHT nanodiamond (ND). ND powder, approximately 500-600 mg of ND powder will be placed in a ceramic boat and inserted into the heating chamber. NDs are oxidized at 525° C. for 5 hours in open air conditions until a tan color is observed. Upon completion of the oxidation process, the NDs are then immediately placed in a glass scintillation vial and stored in a drying oven (−140° C.) to ensure a water free diamond surface. ND—OH samples are alcohol rich and ready to be used for further boron chemistry. Transfer of ND—OH samples should be done under inert conditions into the glovebox environment.

Materials including glassware are placed in a 117° C. drying oven for 24 hours prior to performing any water-sensitive syntheses to ensure complete removal of adsorbed water. Any additional materials such as centrifuge tubes (50 mL polypropylene) and micropipette tips should be placed in a vacuum oven, set at 40° C., 24 hours prior to sample preparation. Trigonal boron molecules, such as boron tribromide and boron trichloride, will be utilized due to their high reactivity and ability to conjugate with the surface of NDs. As a result of this high reactivity, especially to oxygen and water, all syntheses should be performed in an inert nitrogen atmosphere glovebox.

In a typical synthesis, 40 mg of ND—OH is placed inside a dried 100 mL round bottom flask and transferred into the inert atmosphere glove box. Boron tribromide 99% and boron trichloride in 1.0 M in methylene chloride were reacted with ND—OH inside the round bottom flask. To 40 mg ND—OH, 20.8 mL of anhydrous DCM and 0.62 mL BBr₃ was added, capped with a septum, removed from the glovebox and immediately sonicated. Immediately after the addition of the DCM and B—R₃, the reaction vessel (centrifuge tube) is removed from the glove box and sonicated. For sonication, a cup horn sonicator (Fisher Scientific FB505) at 75% of its full power function of 500W was used for two minutes to help solubilize the colloid. Following cup horn sonication, a bath sonicator is used to further solubilize the colloid at 40° C. for 10 min.

The solubilized mixture is to be centrifuged at 5,000 rpm for 25 minutes until the trigonal boron terminated FNDs forms a pellet. The supernatant is then decanted into waste and analyzed via Dynamic Light Scattering (DLS) to ensure minimal loss of FND. Purification cycles are performed in triplicate by adding approximately 10 mL of DCM to the centrifuge tube containing the FND pellet; the sample is once again sonicated and centrifuged to purify the pellet from the unreacted B—R₃. After the final purification cycle, the boron terminated FND samples should be dried using a vacuum pump with a solvent trap inside the inert atmosphere box.

To synthesize multilamellar liposomes suitable for drug loading, a water/oil/water emulsion is prepared. Initially, 25 mg of soybean based asolectin, 1.5 mg of poly(maleic anhydride-alt-1-octadecene) or PMAO is added to a 5 mL centrifuge tube along with 20 μg Cy5 fluorescent dye and 700 μg of chloroform. 200 μg of the boron-ND complex is dispersed in 200 μg of water and added to the same Eppendorf tube. The mixture is then probe sonicated in a 4° C. ice bath at 21% power (1 second of sonication followed by 1 second of rest). After sonication, 2 ml of a 0.5 mg/mL water solution of polyethylene glycol (PEG) is rapidly added to the mixture, the sonicated once more for 1 min. The mixture is then stirred for approximately 12 hours until the suspension turns clear and the chloroform has evaporated from the emulsion. The resulting product is then dialyzed with distilled H₂O overnight to produce drug-loaded multilamellar liposomes. To further purify the multilamellar structures, an extruder coupled with a 100-200 nm filter can be used to obtain unilamellar liposomes to encapsulate the nanodiamond structures.

| Material Name | Material Source | Specifications | Quantity | | Unit Cost |
|---|---|---|---|---|---|
| HPHT nanodiamond | Microdiamant, USA | MSY 0-0.03 micron and | 50 | gr | $10/gram |
| powder | | MSY 0-0.05 micron | | | |
| Anhydrous | Sigma-Aldrich | ≥99.8% Product #270997 | 2000 | ml | $118.00/unit |
| Dichloromethane | | | | | |
| Boron Tribromide | Sigma-Aldrich | ≥99% Product #230367 | 25 | g | $91.60/unit |
| Boron Trichloride | Sigma-Aldrich | 1.0M in methylene | 100 | ml | $94.50/unit |
| | | chloride Product | | | |
| | | #178934 | | | |
| Borane | Sigma-Aldrich | 1.0M in Tetrahydrofuran | 100 | ml | $56.20/unit |
| | | Product #176192 | | | |
| Copper, TEM Grids | Ted Pella, Inc. | 400 mesh, with | 25 | units | $4.00/unit |
| | (Redding, CA) | Ultrathin Lacey | | | |
| | | Carbon Film | | | |
| | | Product #01824 | | | |
| Gold Silica wafers | LGA Thin Films, Inc. | 4-inch silicon wafers | 25 | units | $0.16/unit |
| | (Santa Clara, CA) | coated with a 10 nm | | | |
| | | titanium adhesion layer | | | |
| | | and 100 nm of gold | | | |
| Potassium Bromide | Thermofisher | Spectra tech | 100 | packets | $2.17/packet |
| | Scientific | Product #0016031 | | | |
| Nitrogen (liquid | N/A | N/A | ~1000 | kg | $19.56/kg |
| and gas) | | | | | |
| Soybean azolectin | Sigma-Aldrich | Mixture of soybean | 50 | gr | $56.50 |
| | | phospholipids | | | |
| | | Product #11145 | | | |
| PMAO | Sigma-Aldrich | >99% product | 1 | kg | $170/kg |
| | | number #776866 | | | |
| Chloroform | Sigma-Aldrich | anhydrous, ≥99%, | 1000 | mL | $94.30 |
| | | contains 0.5-1.0% | | | |
| | | ethanol as stabilizer | | | |
| Polyethylene | Sigma-Aldrich | PEG 400 for synthesis | 1 | pack | $27.50 |
| Glycol | | Product #8.07485 | | | |

Data Analysis & Results

Drifts Data

Mechanistically speaking, it was hypothesized that that the highly reactive trigonal boron compounds such as BBr₃ attack the hydroxylated surface of the nanodiamond core. This results in the formation of soluble diatoms such as Br₂ or Cl₂, as well as the sought-after B—C surface bond in which the remaining halide is ripped from the boron and forms soluble weak acids such as ClOH and BrOH. In order to confirm the presence of the B—C bond, diffuse reflectance fourier infrared transform spectroscopy (DRIFTS) was used to analyze the vibrational modes of the oxidized sample. This was confirmed by the presence of have a significant pronounced peak at 1105 wavenumbers corresponding to the C—O surface bonds. We also observed O—H bending at around 1640 wavenumbers, as well as a broad O—H stretch from 3000-3500 wavenumbers. Furthermore, a smaller carboxylic acid peak at 1800 wavenumbers was observed. After the synthesis is complete, we observe a significant shift of the 1100 wavenumbers C—O peak towards about 1025 wavenumbers which corresponds to a B—C surface bond. DRIFTS was used to confirm successful templating of boron onto nanodiamond cores.

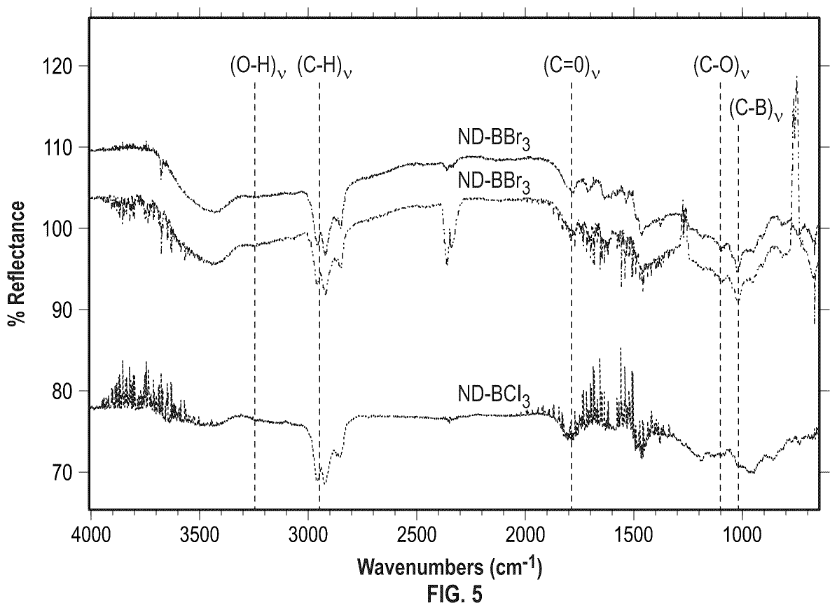

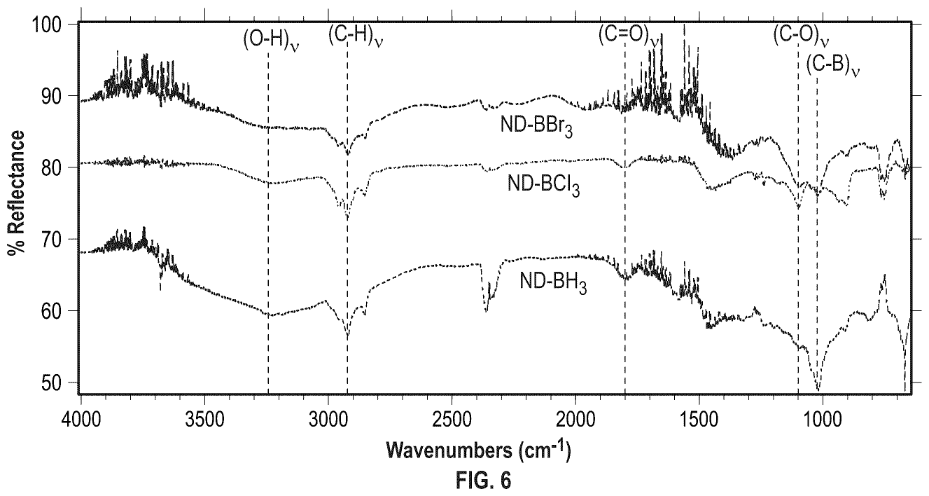

Samples were reacted with a variety of trigonal boron precursors including BBr₃, BCl₃ and BH₃ for varying lengths of time. It was hypothesized that increasing the reaction time from 30 min to 180 min (FIG. 5), to 24 hours (FIG. 6 would have a measurable effect on the extent of boron templating. Although DRIFTS was able to confirm the presence of C—B bonds, further data was needed to quantify the extent of boron templating on the nanodiamond core.

Sem, Tem & Eds Data

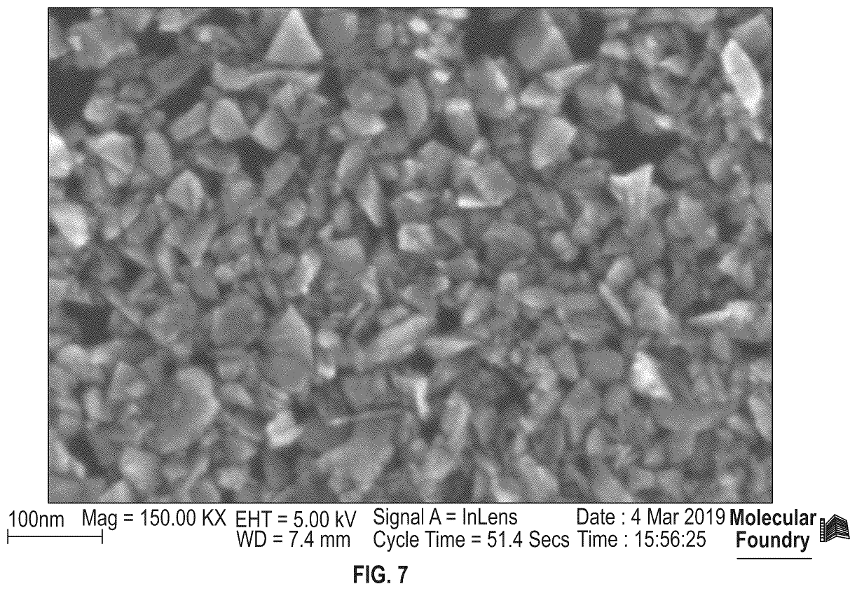

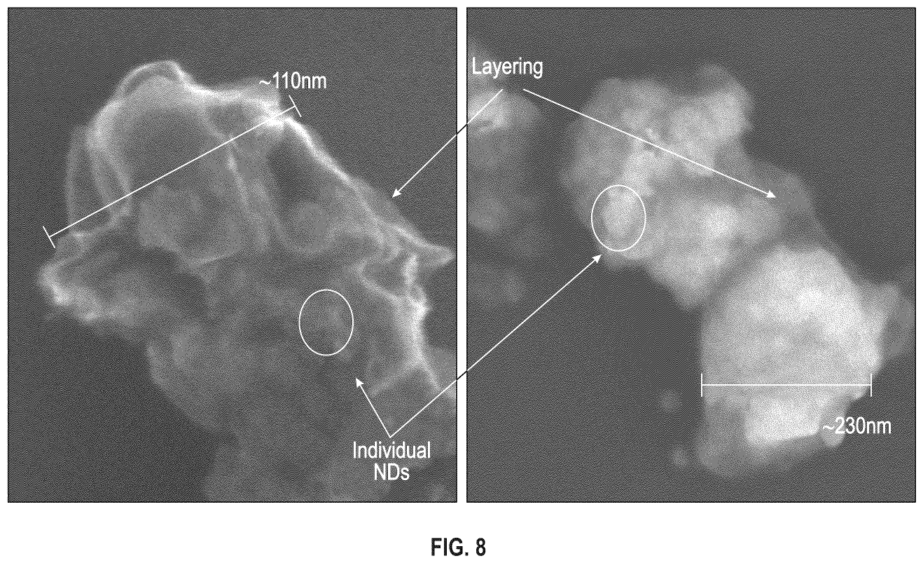

TEM and SEM images were also collected on boron templated nanodiamond samples as further confirmation that templating was successful. TEM data provided some qualitative confirmation that the synthetic process was modifying the diamond surface. When compared to unreacted oxidized nanodiamond controls (FIG. 7), there is a significant change in morphology of the diamonds that had been treated with boron precursors (FIG. 8).

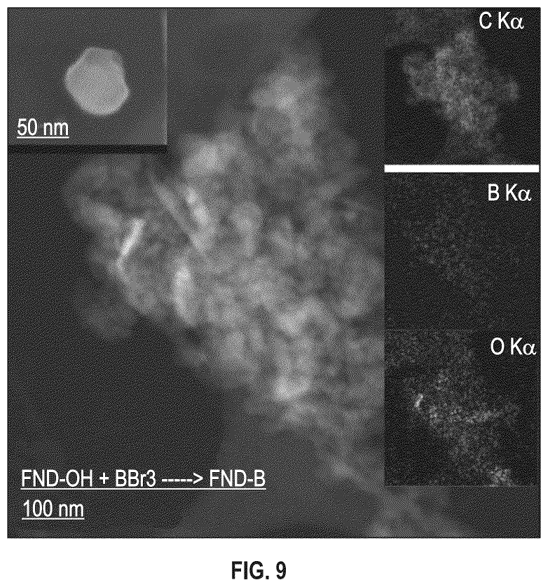

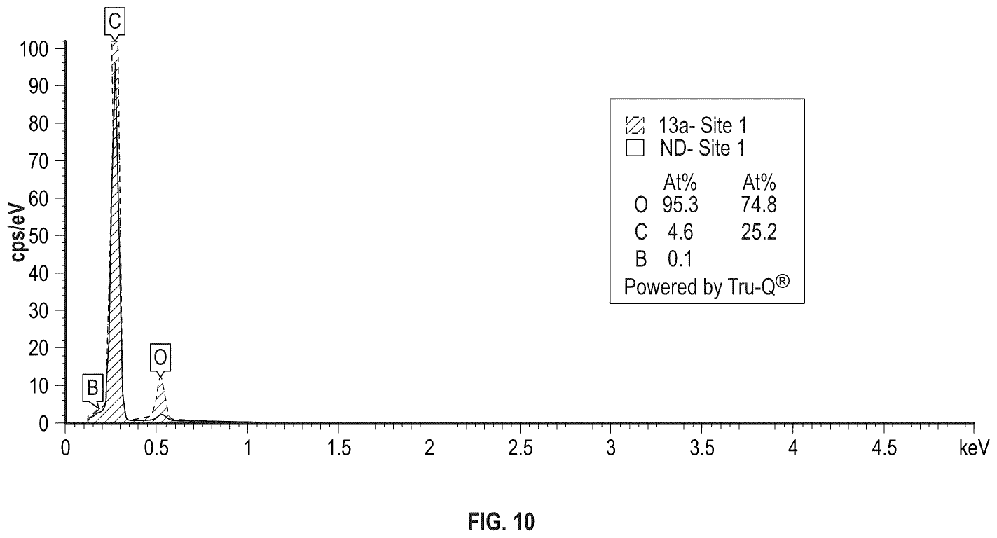

To further quantify the extent of boron templating, SEM and EDS data were collected. EDS data was used to begin to understand the extent of templating and quantify the atomic composition of boron at the nanodiamond surface. EDS data was notoriously difficult to collect due to difficulty resolving boron and carbon signals (FIG. 10). EDS color mapping did show that there was some degree of boron present on the sample surface (FIG. 9). It had been hypothesized that varying the reaction time or increasing the concentration of boron reagents used in the synthesis would have an effect on the extent of templating; to assess the extent of templating quantitatively, more XPS and XAS data was collected.

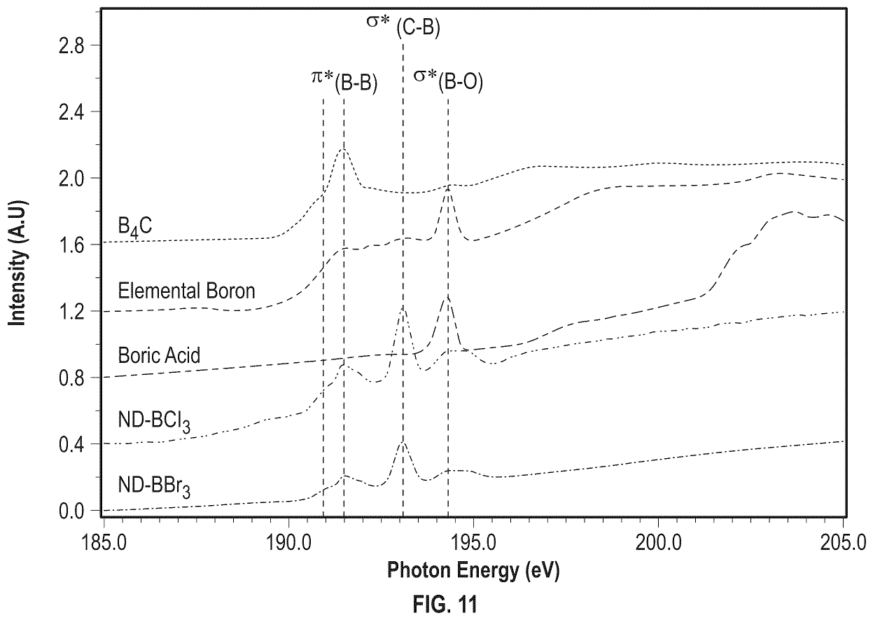

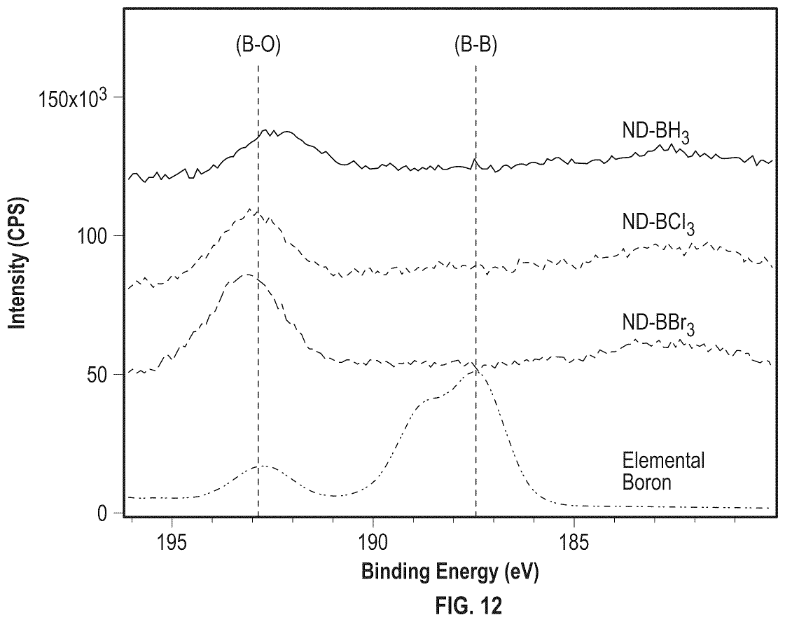

It was initially hypothesized that varying reagent concentration or increasing the reaction time would result in larger shells of boron at the ND surface. XPS data as well as XAS collected to map the elements on our samples indicated otherwise. Spectroscopic analysis seemed to indicate that a boron oxide was present in our samples as opposed to a boron shell coating. In almost all samples, XPS and XAS data showed prominent signals for boron oxide regardless of which reagent was used or how much time the samples were allowed to react (FIG. 11). Although SEM images indicate there is a significant change in morphology following treatment with boron precursors, XPS and EDS data seems to indicate the presence of a thin boron oxide layer. This is contrary to our hypothesis that there was more boron templating in samples that were reacted for longer periods of time.

Liposome Characterization

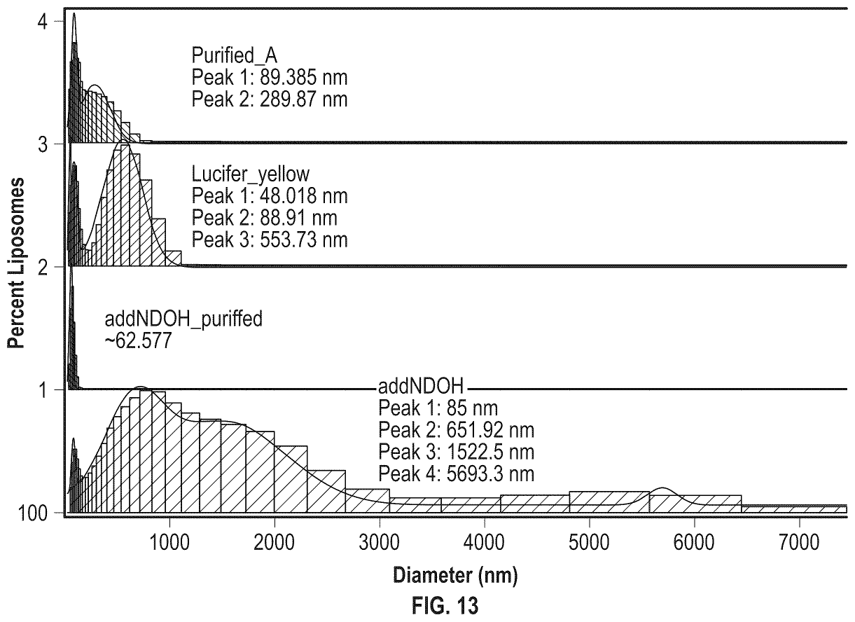

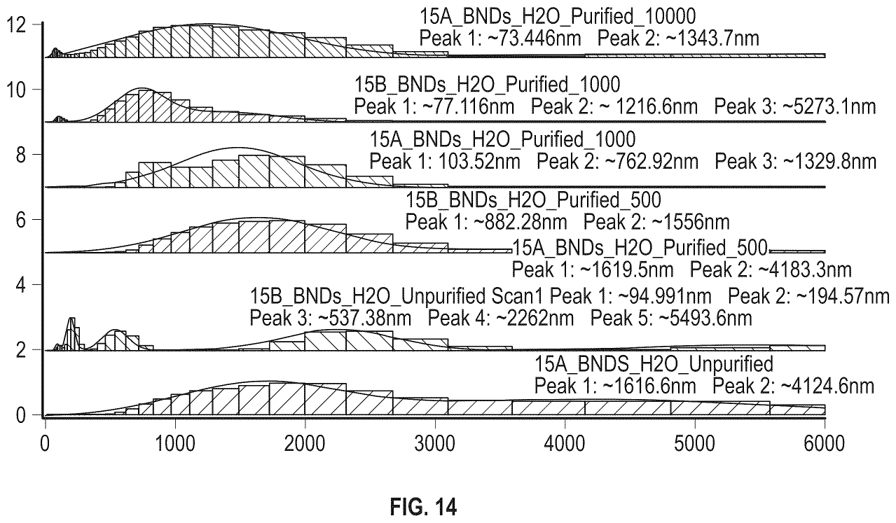

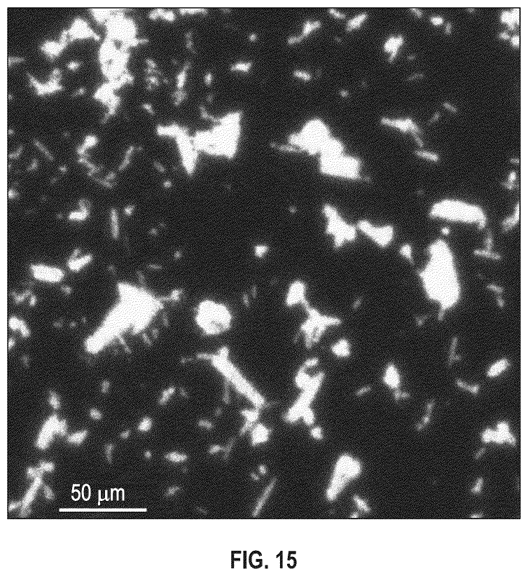

After confirmation that boron templating was successful, work on liposome fabrication began. Liposomes were first synthesized without boronated NDs, and unreacted oxidized nanodiamonds. DLS data was collected to determine distribution of particle size in fabricated liposomes. DLS data collected indicated a particle size that was less than 100 nm which was indicative that the emulsion contained some free nanodiamonds, micelles, or empty liposomes. Purification was performed by extrusion and drastically decreased the polydispersity of the sample (FIG. 13).

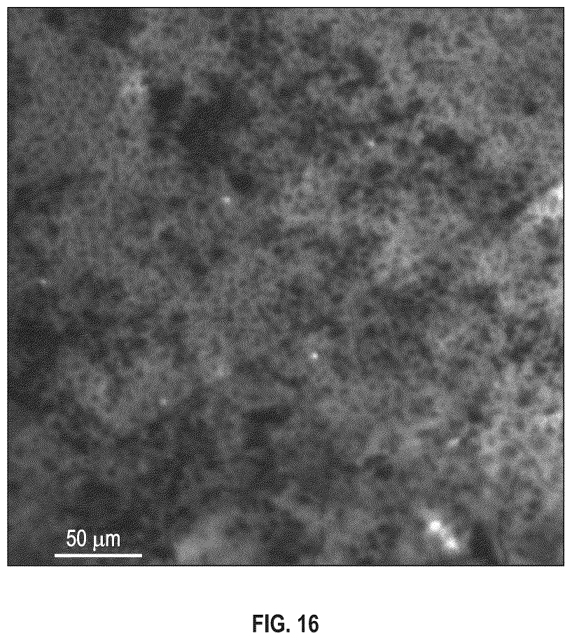

Liposomes were treated with a fluorescent dye Lucifer Yellow and imaged using a confocal microscope. Confocal images show spherical structures as well as dark spots hypothesized to be free B-NDs or unoccupied space (FIG. 16).

Trigonal-planar boranes are proven to modify hydroxyl terminated diamond surface chemistry. The reactivity of these compounds with the ND surface can be directly correlated to the size and electronegativity of the —R group on the borane. Further analysis will be needed to determine the true composition of the surface structures being formed and to gain a better understanding of how organoborane functionalization of FNDs affects the fluorescence on the NV center and how to better control the extent of boron templating. Future work will focus on exploiting this method of manipulating the trigonal planar geometry of the organoboranes decoration of stabilizing and bioreactive species. Catalysts can also be used to enhance the extent of boron coatings to ensure that thicker shells can be templated on the nanodiamond cores. Liposome fabrication was analyzed via DLS and fluorescent microscopy to confirm that boronated FNDs can be encapsulated in unilamellar liposomes. This improves the chance that nanoparticles templated with boron layers encapsulated by liposomes can become attractive vectors for the delivery of ¹⁰B to tumor sites, and ultimately replace older generation BNCT agents such as boron clusters or substituted amino acids.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Formation of Unique Boron Bonds on the Chemically Inert Diamond Surface Using Trigonal Boron Compounds at Room Temperature

25 nm nanodiamonds are oxidized at high temperature in open air which leads to the pyrolysis of amorphous black carbon and OH surface termination of the diamond, yielding a tan powder material that is highly hydrophilic. Diffuse reflectance Fourier infrared transform spectroscopy (DRIFTS) was used to analyze the surface vibration modes of the NDOH sample which was confirmed to have a highly alcohol-rich surface. We observed a significant pronounced peak at 1105 cm⁻¹ corresponding to the C—O surface bonds, we also observed O—H bending at 1640 cm⁻¹, as well as a broad O—H stretch from 3000-3500 cm⁻¹, both likely due to adsorbed water. Furthermore, a smaller carboxylic acid peak at 1785 cm⁻¹ was observed. The O—H stretch and bend were both confirmed to be resulting from water by the implementation of temperature controlled DRIFTS in which we observed the desorption of surface water by 72% at an experimental temperature of 100° C.; by the time the sample is elevated to 200° C. over 97% of the OH bend and stretch that correspond to adsorbed water are gone. Since our starting materials (trigonal boron compounds) are sensitive to water, especially the boron trihalides, we use the peak at 1640 cm⁻¹ to approximately quantify the efficacy of water desorption from our sample. If a significant quantity of water is observed in the raw NDOH sample, we dry the sample at 120° C. under vacuum overnight to ensure that most of the adsorbed water is removed and will not result in a degradation of our starting chemicals, which could lead to side products and even potentially cause the whole boronation/borylation synthesis to fail. After the synthesis is complete, we observe a significant shift of the 1105 cm⁻¹ C—O peak towards about 1025 cm⁻¹ which corresponds to a B—C surface bond. In previous experiments we observed that samples still containing adsorbed water formed far fewer B—C bonds and instead formed soluble borates such as BOH which were then washed away in purification steps. In cases where the NDOH sample was not well dried we actually see a convergence of both B—C and C—O peaks in the 1000-1100 cm⁻¹ area of the spectrograph; the drier the sample, the more dominant the B—C peak.

The formation of B—C bonds on the surface of the NDOH was accomplished by the room temperature reaction of boron trihalides (BBr₃ and BCL₃) with NDOH under inert conditions at 0.302 M BX₃ and 0.777 mg/mL NDOH. We have observed that the synthesis itself is highly sensitive to air and water because the starting chemicals degrade readily in non-inert conditions, on the other hand, the final product formed shows stability of B—C bonds when exposed to air and water. Further research will explore the thermal stability of these bonds using temperature controlled DRIFTS and will hopefully enable us to gain a better understanding of the reaction mechanism.

### Exemplary Aspects

The following exemplary aspects are provided, the numbering of which is not to be construed as designating levels of importance:

Aspect 1 provides a functionalized nanoscale substrate comprising:
a functionalized surface of the substrate comprising:
a boronated portion.

Aspect 2 provides the functionalized nanoscale substrate of Aspect 1, wherein the boronated portion comprises a ¹⁰B boron.

Aspect 3 provides the functionalized nanoscale substrate of any one of Aspects 1 or 2, wherein the substrate comprises diamond, gold, silver, silica or a mixture thereof.

Aspect 4 provides the functionalized nanoscale substrate of any one of Aspects 1-3, wherein the substrate comprises diamond

Aspect 5 provides the functionalized nanoscale substrate of any one of Aspects 1-4, wherein the boronated portion is disposed over about 40 to about 100 percent of a total surface area of the surface of the substrate.

Aspect 6 provides the functionalized nanoscale substrate of any one of Aspects 1-5, wherein the boronated portion is disposed over about 60 to about 100 percent of a total surface area of the surface of the substrate.

Aspect 7 provides a functionalized nanoscale substrate comprising:
a boronated surface comprising a ¹⁰B boron, wherein the nanoscale substrate comprises diamond.

Aspect 8 provides the functionalized nanoscale substrate of any one of Aspects 1-7, wherein a major dimension of the functionalized nanoscale substrate is in a range of from about 5 to about 100 nm.

Aspect 9 provides the functionalized nanoscale substrate of any one of Aspects 1-8, wherein a major dimension of the functionalized nanoscale substrate is in a range of from about 15 to about 80 nm.

Aspect 10 provides a delivery vehicle comprising:
a liposome encapsulating the functionalized nanoscale substrate of any one of Aspects 1-9.

Aspect 11 provides a method of making the functionalized nanoscale substrate of any one of Aspects 1-10, the method comprising:
boronating a hydroxylated nanoscale substrate.

Aspect 12 provides the method of Aspect 11, wherein boronating the hydroxylated nanoscale substrate comprises reacting the hydroxylated nanoscale substrate with boron tribromide, boron trichloride, or a mixture thereof.

Aspect 13 provides a method of making the delivery vehicle of Aspect 10, the method comprising:
contacting the functionalized nanoscale substrate with one or more lipids; and
mixing the functionalized nanoscale substrate with one or more lipids to form one or more liposomes encapsulating the functionalized nanoscale substrate.

Aspect 14 provides the method of Aspect 13, wherein the one or more lipids comprises one or more phospholipids

Aspect 15 provides a method of treating a tumor, the method comprising:
administering a therapeutically effective amount of the functionalized nanoscale substrate to a patient in need thereof;
contacting the administered functionalized nanoscale substrate with electromagnetic radiation.

Aspect 16 provides the method of Aspect 15, wherein functionalized nanoscale
substrate is encapsulated in a liposome.

Aspect 17 provides the method of any one of Aspects 15 or 16, wherein the tumor comprises a brain tumor.

Aspect 18 provides the method of any one of Aspects 15-17, wherein the tumor comprises a cutaneous melanoma or a glioblastoma.

What is claimed is:

1. A functionalized nanoscale substrate comprising:
a functionalized surface of the substrate comprising:
a boronated portion.

2. The functionalized nanoscale substrate of claim 1, wherein the boronated portion comprises a ¹⁰B boron.

3. The functionalized nanoscale substrate of claim 1, wherein the substrate comprises diamond, gold, silver, silica or a mixture thereof.

4. The functionalized nanoscale substrate of claim 1, wherein the substrate comprises diamond

5. The functionalized nanoscale substrate of claim 1, wherein the boronated portion is disposed over about 40 to about 100 percent of a total surface area of the surface of the substrate.

6. The functionalized nanoscale substrate of claim 1, wherein the boronated portion is disposed over about 60 to about 100 percent of a total surface area of the surface of the substrate.

7. A functionalized nanoscale substrate comprising:
a boronated surface comprising a ¹⁰B boron, wherein the nanoscale substrate comprises diamond.

8. The functionalized nanoscale substrate of claim 7, wherein a major dimension of the functionalized nanoscale substrate is in a range of from about 5 to about 100 nm.

9. The functionalized nanoscale substrate of claim 7, wherein a major dimension of the functionalized nanoscale substrate is in a range of from about 15 to about 80 nm.

10. A delivery vehicle comprising:
a liposome encapsulating the functionalized nanoscale substrate of claim 1.

11. A method of making the functionalized nanoscale substrate of claim 1, the method comprising:
boronating a hydroxylated nanoscale substrate.

12. The method of claim 11, wherein boronating the hydroxylated nanoscale substrate comprises reacting the hydroxylated nanoscale substrate with boron tribromide, boron trichloride, or a mixture thereof.

13. A method of making the delivery vehicle of claim 10, the method comprising:
contacting the functionalized nanoscale substrate with one or more lipids; and
mixing the functionalized nanoscale substrate with one or more lipids to form one or more liposomes encapsulating the functionalized nanoscale substrate.

14. The method of claim 13, wherein the one or more lipids comprises one or more phospholipids

15. A method of treating a tumor, the method comprising:
administering a therapeutically effective amount of the functionalized nanoscale substrate to a patient in need thereof;
contacting the administered functionalized nanoscale substrate with electromagnetic radiation.

16. The method of claim 15, wherein functionalized nanoscale substrate is encapsulated in a liposome.

17. The method of claim 15, wherein the tumor comprises a brain tumor.

18. The method of claim 15, wherein the tumor comprises a cutaneous melanoma or a glioblastoma.
